(19) United States
(12) Patent Application Publication
(10) Pub. No.: US 2008/0269845 A1
(43) Pub. Date: Oct. 30, 2008

(54) WAVEGUIDE CONNECTION DEVICE

(75) Inventors: Andreas Rose, San Marcos, CA (US); Kyle Johnston, Sammamish, WA (US); Guenter Herr, Ehringshausen (DE)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 11/876,376

(22) Filed: Oct. 22, 2007

Related U.S. Application Data
(60) Provisional application No. 60/914,638, filed on Apr. 27, 2007.

(51) Int. Cl. A61N 5/06; G02B 6/38

(52) U.S. Cl. 607/88; 385/58

(57) ABSTRACT

A connector for interconnecting a waveguide with a light component (e.g., another waveguide) for allowing light communication therebetween. Assemblies for connecting a waveguide can be formed with the connector. In one embodiment, the connector is employed for connecting to a waveguide useful in performing medical applications such as provision of photodynamic therapy.

### CLAIM OF BENEFIT OF FILING DATE

This application claims the benefit of the filing date of U.S. Provisional Application Ser. No. 60/914,638 filed on Apr. 27, 2007, and incorporated herein by reference for all purposes.

### FIELD OF INVENTION

The present invention relates to waveguide connection devices, assemblies formed with the connection devices and uses for the assemblies and devices such as providing communication between waveguides that provide light for photodynamic therapy.

### BACKGROUND OF THE INVENTION

There exist numerous situations in which it can be desirable to interconnect one or more waveguides to another waveguide or to other components for providing light communication between the waveguides and/or between a waveguide and another component (e.g., an optical element, light source detector or the like). It can be desirable to provide such interconnection for automotive applications, medical applications, data transfer applications or the like. One particular application, photodynamic therapy provides various situations where it is desirable to provide such connection.

Interconnecting to waveguides can be problematic. It can be difficult to connect a waveguide to another component (e.g., another waveguide or other component) in a manner that properly aligns the waveguide with the other component such that there is low light loss as light is transferred from between the waveguide and the other component. It can be difficult to provide secure attachment of a waveguide to a connection device. It can also be difficult to create a connection that is easy to engage and/or disengage, particularly if it is desirable for the connection to be operable with one hand. Accordingly, there is a need to create a connection for waveguides that addresses one or more of these difficulties or other difficulties. There is also a need to create an assembly suitable for performing photodynamic therapy where the assembly allows for desirable connection and/or release of one or more waveguides.

### SUMMARY OF THE INVENTION

Accordingly, there is provided a connector and assemblies that include the connector. The assemblies typically include one or more of a first waveguide or other light component, a second waveguide, a light source and/or a light distribution device in conjunction with the connector. Typically, the connector is connected to the first waveguide or other light component. When included, the first waveguide typically includes a first optical fiber. The connector is also typically connectable to the second waveguide. The second waveguide can include a second optical fiber and that optical fiber can be disposed within a covering. The second optical fiber is typically formed of a polymeric material and, when present, the covering is also formed of a polymeric material. One preferred polymeric material is polyethylene. The connector has a first feature for connecting to the first waveguide or other light component and a second feature for connecting to the second waveguide. The first feature can include an opening into which an end of the first waveguide extends such that the end of the first waveguide can be securely located within the opening of the first feature by virtue of an interference fit between the connector and the first waveguide. The second feature typically includes an opening into which an end of the second waveguide extends. The second feature preferably includes a clamping member that clamps the second waveguide between a first surface and a second surface of the connector for aligning the second optical fiber with the first optical fiber or other light component such that light can be communicated therebetween. In one embodiment, the clamping member has an axis, which preferably extends laterally with respect to an axis of the connector. Preferably, in such embodiment, movement of the clamping member along its axis causes the first surface of the connector to move closer and/or further away from the second surface of the connector such that the second optical fiber is securely, but releasably, clamped between the first surface and the second surface.

### BRIEF DESCRIPTION OF THE DRAWINGS

The features and inventive aspects of the present invention will become more apparent upon reading the following detailed description, claims, and drawings, of which the following is a brief description:

FIG. 1 is a side sectional view of an exemplary assembly according to the present invention;

FIG. 2 is a side view of an exemplary connector that is being employed in the assembly of FIG. 1;

FIGS. 3A and 3B are end views of the exemplary connector of FIG. 1;

FIG. 4 is perspective view of an exemplary assembly that includes the of FIG. 1 connected to an exemplary second waveguide and an exemplary light distribution device;

FIG. 5 is a sectional view of another exemplary connector according to another aspect of the present invention;

FIGS. 6A and 6B are respectively a sectional view and an end view of another exemplary connector according to another aspect of the present invention;

FIG. 7 is a sectional view of another exemplary connector according to another aspect of the present invention;

FIGS. 8A and 8B are respectively a sectional view and a top cut-away view of another exemplary connector according to another aspect of the present invention;

FIGS. 9A and 9B are respectively a side sectional and a front sectional view of another exemplary connector according to another aspect of the present invention; and

FIG. 10 illustrates another exemplary connector in accordance with an aspect of the present invention.

### DESCRIPTION OF THE PREFERRED EMBODIMENT

The present invention is predicated upon the provision of a connector for interconnecting a waveguide with another light component (e.g., a light source, another waveguide or the like) in a manner that allows for light communication therebetween. The present invention also provides methods of interconnecting a waveguide to another light component or waveguide for particular uses as well as assemblies suitable for such uses. It is contemplated that the connector, the methods of connection and the assemblies of the present invention can be used for a variety of purposes and/or articles of manufacture such as automotive vehicles, detectors, sensors, lenses, instruments such as a spectrograph, or the like. However, the connector, the methods of connection and the assemblies of the present invention have been found particularly useful for medical applications such as provision of photodynamic therapy.

The connector of the present invention typically includes a connector body, a first attachment feature for attaching to a first waveguide and second attachment feature for attaching to a second waveguide. Preferably, the first feature and the second feature respectively includes a first opening for receiving a first waveguide and a second opening for receipt of a second waveguide. The connector also typically includes a clamping member associated with the connector body where the clamping member at least assists in attaching the connector to the second waveguide and/or aligning the second waveguide with the first waveguide. Preferably the clamping member can move a first surface of the connector toward and/or away from a second surface of the connector such that the second waveguide can be securely but releasably attached to the connector.

I. Definitions

The following terms are intended to have the following general meanings as they are used herein:

Body cavity: any cavity within a body such as ear, nose, vagina, lung, the entire digestive track (e.g., throat, esophagus, stomach, intestines, rectum, etc.), gall bladder, bladder, any open wound or the like. The body cavity can be within a human body or a body of another animal.

Light Component: a device that can produce light, transfer light or detect light such as a light source (e.g., a laser), a detector, an LED or the like.

Light: light at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally between about 100 nm to 10,000 nm, with exemplary ranges between about 160 nm to 1600 nm, between about 400 nm to about 900 nm, and between about 500 nm to about 850 nm, although the wavelengths may vary depending upon the particular photosensitizing compound used and the light intensity. Depending on the application, the light produced may be a single wavelength or multiple wavelengths. The light may be produced by any suitable art-disclosed light emitting devices such as lasers, light emitting diodes (“LEDs”), arc lamps, incandescent sources, fluorescent sources, gas discharge tubes, thermal sources, light amplifiers or the like.

Light Source: a light emitting device such as laser, light emitting diode (“LEDs”), arc lamp, incandescent source, fluorescent source, gas discharge tube, thermal source, light amplifier, or a combination thereof. The output of the light source is preferably adjustable so that the operator can modify the wavelength, the power output, the size of illumination, or combinations thereof while carrying out the present method. For example, the wavelength of a laser may be adjusted to activate different photosensitizers in the photosensitizing composition. Alternately, the power of the light source may be increased or decreased after an application of light energy to the treatment area. In addition, the light source may comprise a temperature monitoring device so that over heating of the host tissues in and around the treatment area may be avoided. Suitable temperature monitoring devices may comprise an IR device, a fiber optic device, a thermocouple, or a combination thereof.

Microbes: any and all disease-related microbes such as virus, fungus, and bacteria including Gram-negative organisms, Gram-positive organisms or the like. Some examples of microbes include but are not limited to, Staphylococcus aureus, Methicillin-resistant Staphylococcus aureus (“MRSA”), Escherichia coli (“E. coli”), Enterococcus fecalis (“E. fecalis”), Pseudomonas aeruginosa, Aspergillus, Candida, etc.

Photosensitizing composition: a composition comprising at least one suitable art-disclosed photosensitizer that has at least an antimicrobial action upon application of electromagnetic energy of certain wavelength(s). Suitable photosensitizers include both Type I and Type II photosensitizers, where Type I photosensitizers produce a free radical upon the application of light and Type II photosensitizers produce singlet oxygen upon the application of light. While photosensitizers that have other modes of operation (e.g. generation of heat) are contemplated, those types discussed above are preferred. Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. Patent Publication No. 2003-0180224. Preferred phenothiazines include methylene blue (MB), toluidine blue (TBO), and those discussed in U.S. Patent Publication No. 2004-0147508. Other preferred antimicrobial photosensitizers include indocyanine green (ICG). Combinations of two or more photosensitizers, such as MB and TBO or the like, are also suitable. The photosensitizer may be present in the photosensitizer composition in any suitable amounts. Examples are between about 0.001 percentage of total weight (wt %) and 10 wt %, between about 0.005 wt % and about 1 wt %, between about 0.01 wt % to about 0.5 wt %, and between about 0.02 wt % to about 0.1 wt %. The photosensitizing composition may optionally contain a therapeutic agent, which is any chemical, drug, medication, proteinaceous molecule, nucleic acid, lipid, antibody, antigen, hormone, nutritional supplement, cell or any combination thereof that helps ameliorate a condition. Preferred therapeutic agents include those that promote wound healing, have antimicrobial action, have anti-inflammatory action, and/or provide pain relief. The photosensitizing composition may also optionally contain carriers, diluents, or other solvents for the photosensitizer or other components of the composition and may be used to adjust the concentration of photosensitizer. The photosensitizing composition may be any suitable phase such as a liquid, gel, paste, putty, or solid. Preferably, the compositions has a viscosity low enough to flow into the treatment site while also having a viscosity high enough to maintain the composition within the treatment site. Further compositions that become liquid after application to the treatment site are contemplated such as those that melt or go into solution in the treatment site. Alternately, the composition may gel after application to the treatment site as a liquid; this would permit the composition to cover the treatment site effectively, while also maintaining the composition in the treatment site. The photosensitizers mentioned above are examples and are not intended to limit the scope of the present invention in any way.

Connector

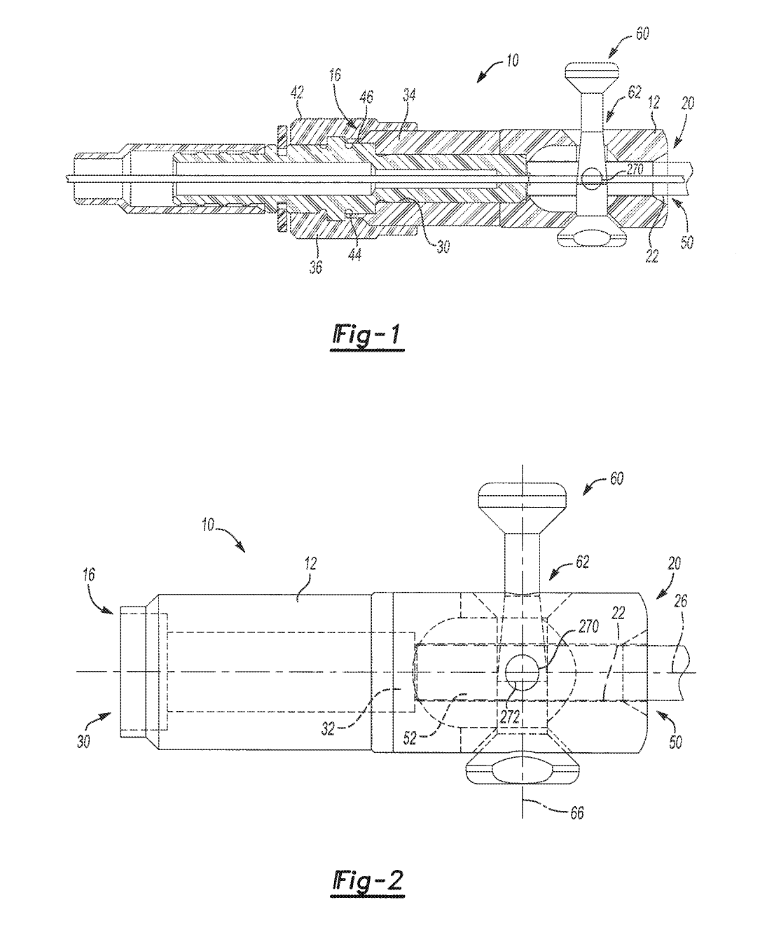

With reference to FIGS. 1-4, there is illustrated an exemplary connector 10 having a body 12, a first feature 16 for attachment to a first waveguide 18 and a second feature 20 for attachment to a second waveguide 22. The connector 10, particularly the body 12 of the connector 10, extends along an axis 26 (e.g., a longitudinal axis). The body 12 is shown as being generally annular but may be alternatively shaped.

The first attachment feature 16 typically includes an opening 30 (e.g., a pocket or cavity) for receiving an end 32 of the first waveguide 18. The first attachment feature 16 also typically includes one or more portions 34 designed to interferingly fit to one or more portions 36 of the waveguide 18. Such interference fits can include mechanical interlocks (e.g., protrusion and cavity interlocks), interlocking threads, friction fits, combinations thereof or otherwise. In the particular embodiment illustrated, a surface of the connector 10, particularly the connector body 12, is friction fit against a surface of an attachment member 42 at the end of first waveguide 18 within an opening 44 (e.g., a cavity) of the attachment 42. Also a surface of another attachment member 46 at the end of the first waveguide 18 can be friction fit against a surface defining the opening 30 of the connector 10. In the embodiment shown, the first waveguide 18 includes a standard SMA fiber attachment and an end of the connector 10 is designed to accommodate the attachment 42. It is contemplated, however, that various alternative mechanisms may be employed to connect the first waveguide to the connector and that the connector can be modified as needed or desired to connect to the first waveguide depending upon the nature of the first waveguide. Preferably, the first waveguide is removable or detachable from the connector for allowing cleaning, sterilization or both of the connector, the first waveguide or both.

The second attachment feature 20 of the connector 10 includes an opening 50 (e.g., a cavity) for receiving an end 52 of the second waveguide 22. In the embodiment shown, that opening 50 extends along the axis 26 of the connector 10 and adjoins the opening 30 of the first attachment feature 16. The second attachment feature 20 also includes a clamping member 60 that is configured to clamp the second waveguide 22 between a first surface and a second surface of the connector 10. In the embodiment shown, the clamping member 60 is located within a further opening 62 (e.g., cavity or through-hole) of the connector 10. As shown, the further opening 62 and the clamping member 60 extend along an axis 66 thereof and that axis 66 extends laterally (e.g., substantially perpendicular) to the axis 26 of the connector 10 itself.

The first and second surfaces of the connector 10 that clamp the second waveguide 22 can be provided by various parts of the connector 10. For example, the clamping member 60 could move components of the connector 10 such that surfaces of those components clamp the second waveguide 22. In the embodiment shown, the first surface 68 is provided as an outer or peripheral surface of the clamping member 60 and the second surface 70 is provided as an interior surface that defines the cavity 50 of the attachment feature 20.

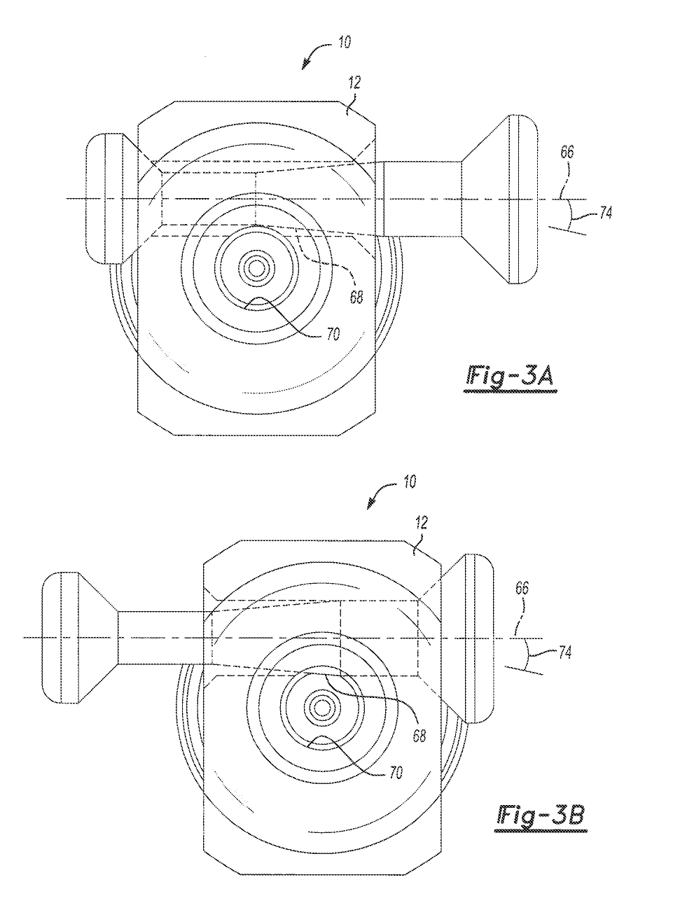

In FIG. 3A, the clamping member 60 is shown in an open or non-clamping position. In this position, the end 52 of the second waveguide 22 can be inserted into the opening 50 of the connector 10, preferably until it abuts a surface (e.g., the bottom surface) of the opening. Thereafter, the clamping member 60 can be moved along its axis 66 such that the surface 68 of the clamping member 60 and the surface 70 of the connector 10 press against the outer surface of the second waveguide 22 thereby clamping the waveguide 22. Such clamping can align the second waveguide 22 with the first waveguide 18 such that light from the first waveguide 18 can be communicated to the second waveguide 22. The connector 10, and particularly the surfaces 68, 70 of the connector 10, are so dimensioned that the axes of the waveguide align upon clamping. It is also preferable for the spacing between the waveguides upon clamping to be such that there is low loss of light in transfer of the light from one waveguide to the other. This may mean that there substantially no space at all between the waveguides and/or that the first waveguide bores into the second waveguide. Further, the ending faces of the second waveguide, the first waveguide or both may be smooth and polished and may be substantially perpendicular to their own or each others axes.

It is generally preferred that one or both of the clamping surfaces be disposed at a first angle relative to the axis of travel of the clamping member. In the embodiment illustrated, the surface 68 of the clamping member 60 is disposed at an angle 74 relative to the axis of travel of the clamping member 60, that axis being the axis 66 of the clamping member 60. The angle 74 is typically at least about 10, more typically at least about 30 and even more typically at least about 5°. The angle 74 is also typically less than about 45° and more typically less than about 25°.

Advantageously, the surface 68 can then more easily clamp the second waveguide 22 as it is moved along the axis 66. The surface 68 can be disposed at such angle using various techniques. In the illustrated embodiment, the cross-sectional area of the clamping member is progressively increased along the axis of the clamping member 60.

The clamping member 60 can also be moved to release or unclamp the second waveguide from between clamping surfaces. In the particular embodiment illustrated, the clamping member 60 is moved along its axis 66 such that the waveguide 22 is released from between the surface 68 of the clamping member 60 and the surface 70 of the connector 10 as those surfaces move away from each other.

When the first waveguide 18 is attached to the connector 10 and the second waveguide 22 is clamped in position, the first waveguide 18 and the second waveguide 18 are preferably aligned such that light can be transferred from the first waveguide 18 to the second waveguide 22 without any substantial loss of light. Preferably, the amount of light lost in that transfer is less than 5%, more typically less than 2% an even possibly less than 0.1%.

Waveguides

It shall be recognized that numerous types of waveguides can be employed for the first waveguide, the second waveguide or both. Exemplary waveguides include, without limitation, silica core/silica cladding optical fibers, silica core/polymer clad optical fibers, polymer optical fibers including plastic optical fibers, multi-core bundles of fibers and photonic crystal fibers. In a preferred embodiment, the first waveguide can be a reusable waveguide while the second waveguide can be a disposable waveguide. Any of the fibers discussed can be reusable waveguides suitable for use as the first waveguide while the choice of disposable waveguide is typically driven by cost.

Disposable fibers or waveguides suitable for use as the second waveguide are typically formed of relatively inexpensive material. Such a material is typically a polymeric (e.g., plastic) material. The fiber can include a jacket, such as a polymeric jacket, that substantially surrounds the fiber along its length. Although various materials can be used for the jacket, one preferred material is a relatively compliant material such as polyethylene. The fiber typically has a diameter that is at least about 0.1 mm and more preferably at least about 0.5 mm. The diameter of the fiber is also typically less than about 5 mm and more preferably less than about 2 mm (e.g., about 1 mm). The fiber with the jacket surrounding it, typically has a diameter of at least about 0.8 mm more typically at least about 1.5 mm. Also, the fiber with the jacket surround it, typically has a diameter of less than about 10 mm and more typically less than about 4 mm (e.g., about 2.2 mm). Preferably, although not required unless otherwise stated, the diameter of the opening 50 that receives the second waveguide 22 is no greater 150%, typically no greater than 120% and even more typically no greater 108% of the diameter of the second waveguide.

Assemblies

The connector and one or both of the first component (e.g., the first waveguide or another component) and second waveguide can be used in a variety of assemblies. Typically, the first component is a light component, which is typically as a light source, a detector or waveguide that is connected to a light source or detector with an attachment. The second waveguide is typically connected to or connectable with a light distribution device. The light distribution device could be a probe, a conical member, a bulb member or the like. The distribution device can be reusable (e.g., through sterilization such as autoclaving). However, in a preferred embodiment, the distribution device is disposable. In such an embodiment the distribution device will typically be formed of a low cost material such as a polymeric (e.g., plastic) material.

The light distribution device can, in one embodiment, be configured for performing photodynamic therapy. In one embodiment, the light distribution device is shaped for insertion into a cavity of a body (e.g., a human body). Generally, for performing photodynamic therapy, a photosensitizing solution can be delivered to tissue of the body within the cavity of the body, the light distribution device can be inserted within the cavity and light can be emitted for killing microbes.

With reference to FIG. 4, an assembly 78 is illustrated to include the connector 10, the first or reusable waveguide 18, a light source 80 and a second or disposable waveguide 22. As shown, the connector 10 connects to the first waveguide 18 and to the second waveguide 22. As such, light can be transmitted from the light source 80 to and through the first waveguide 18 to and through the connector 10 and the second waveguide 22. The second waveguide 22 is connected to a disposable distribution device 84 to which the light is finally transferred. The particular device 84 is a generally bulb shaped plastic member with a generally rounded end that is suitable for insertion into a nasal cavity. The device 84 is designed to distribute light about the nasal cavity for performing photodynamic therapy. Of course, multiple devices like the device of FIG. 4 could be configured as needed to distribute light to locations (e.g., bodily cavities) at which photodynamic therapy is to be performed.

Additions and Alternatives

There are multiple additional or alternative features and/or embodiments that can be used in conjunction with or separately from the features and/or embodiments already described. It shall be understood that the discussions above also apply to these additional or alternative features and/or embodiments as would be understood by the skilled artisan. Moreover, the disclosed additional or alternative features and/or embodiment are intended to be exemplary and not exhaustive.

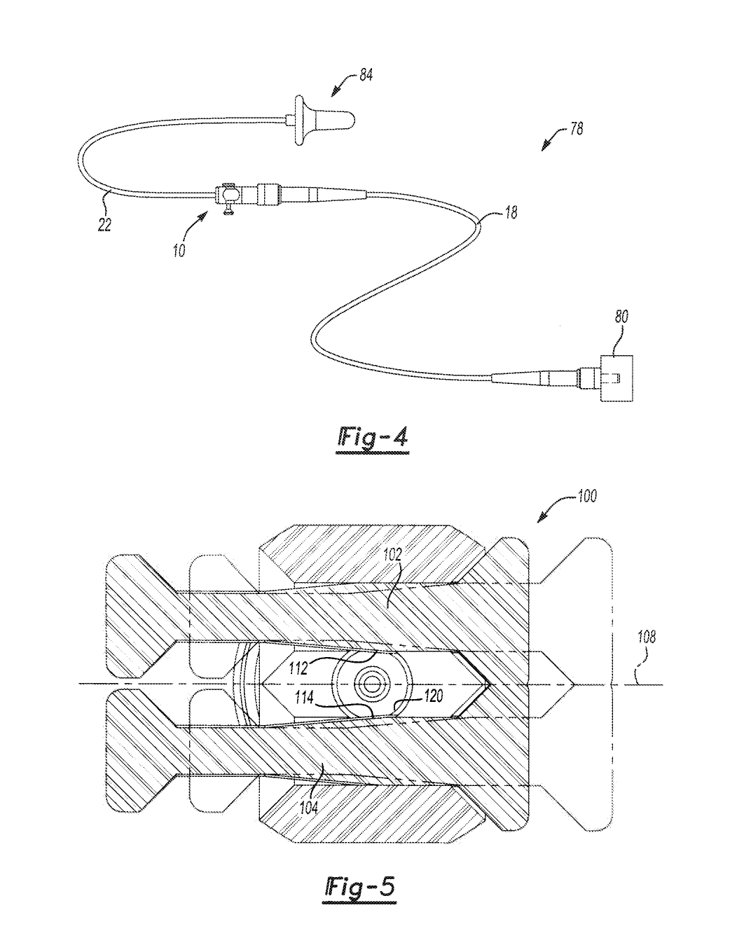

Generally, it is contemplated that the connector of the present invention can be configured with a clamping member having two or more portions with two or more surfaces between which a waveguide can be clamped. With reference to FIG. 5, a clamping member 100 is illustrated to include a first clamping portion 102 (e.g., first clamp bar) and a second clamping portion 104 (e.g., second clamp bar). As shown, the clamping member 100 has an axis 108 of travel and each of the clamping portions 102, 104 includes a clamping surface 112, 114 that is angled relative to that axis 108 in a manner as described with respect to the clamping member 10 of FIGS. 1-4. As the clamping member 100 is moved along the axis 108, it can clamp a waveguide (e.g., a second or disposable waveguide) between the portions 102, 104 and/or surfaces 112, 114 of those portions 102, 104 by pushing the waveguide against a wall or surface 120 of the cavity of the connector.

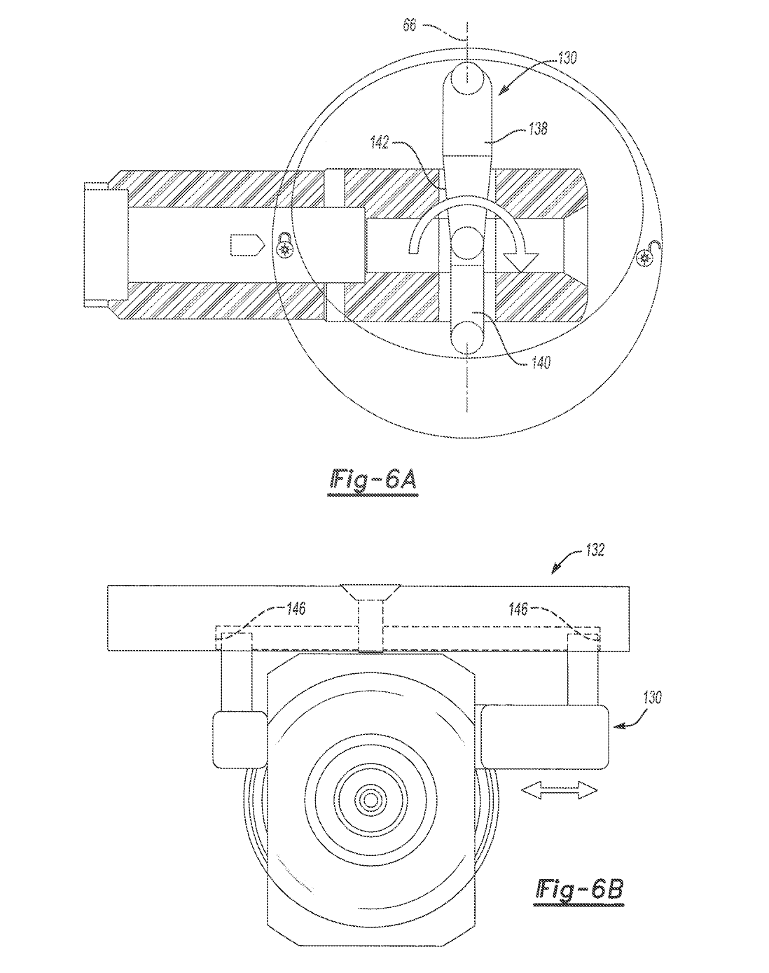

FIGS. 6A and 6B illustrate a clamping member 130 that is connected to a movable member 132 that can assist the clamping member 130 in engaging and disengaging a waveguide such as the second waveguide. The clamping member 130 is illustrated as a having a first portion 138 and a second portion 140 connected to each other with a surface 142 (like the surface 68 of FIGS. 3A and 3B). The clamping member 130 is connected at its ends to the movable member 132, which is a pivotable cam engagement member. The movable member 132 can be pivoted (e.g., by turning the member around its pivot point). As shown in FIGS. 6A and 6B, the cam surface 146 of the movable member 132 is engaged against a protrusion or protrusion[s] at the end[s] of the movable member 132 as the moveable member is pivoted thereby forcing the clamping member 130 to move along its axis 66 until the connector is in an open or unlocked state similar to FIG. 3A. When the movable member is pivoted (e.g., 180°) the cam surface 146 engages at least one of the protrusions extending from the movable member forcing the member to move along its axis 66 to a closed or locked state. In this manner, the clamping member 130 can operate in the same manner as described for the embodiment of FIGS. 1 and 2 to clamp a waveguide.

It is contemplated that the movable member could be oriented so its rotary motion pivots along an axis parallel to the axis of the connector instead of perpendicular. Moreover, for some applications, it would be advantageous for the moveable member to be fitted with a ratcheting feature so that it turns only one direction. Alternatively, the movable member or connector could have stop features to limit the pivoting of the movable member. As shown, the moveable member can have an indicator (e.g., an arrow symbol) that point to an open or closed icon.

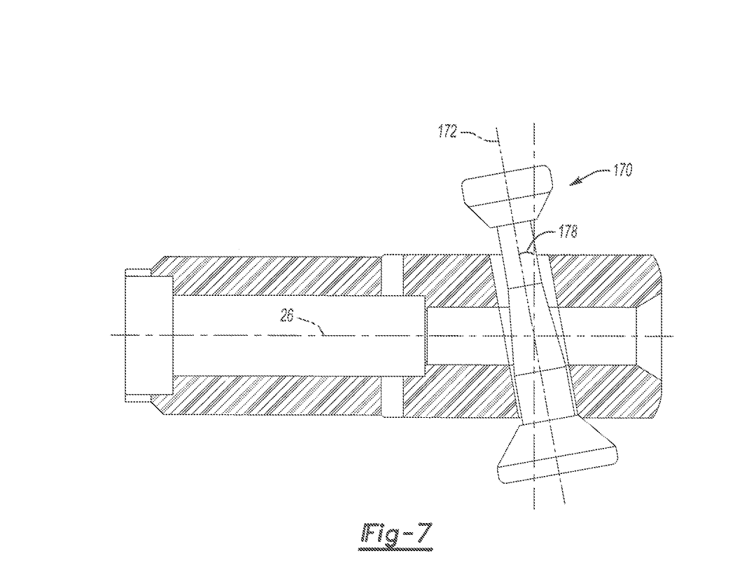

With reference to FIG. 7, it is contemplated that a clamping member 170 may be have an axis 172 and/or be configured to travel along an axis 172 that is angled or non-perpendicular to the axis 26 of the connector, the second waveguide or both. In such an embodiment, the axis 172 of the member 170 and/or travel is typically at an angle 178 of between about 1° and about 30°, more typically between about 5° and about 20° relative to an axis that is perpendicular to the axis of the connector and/or waveguide.

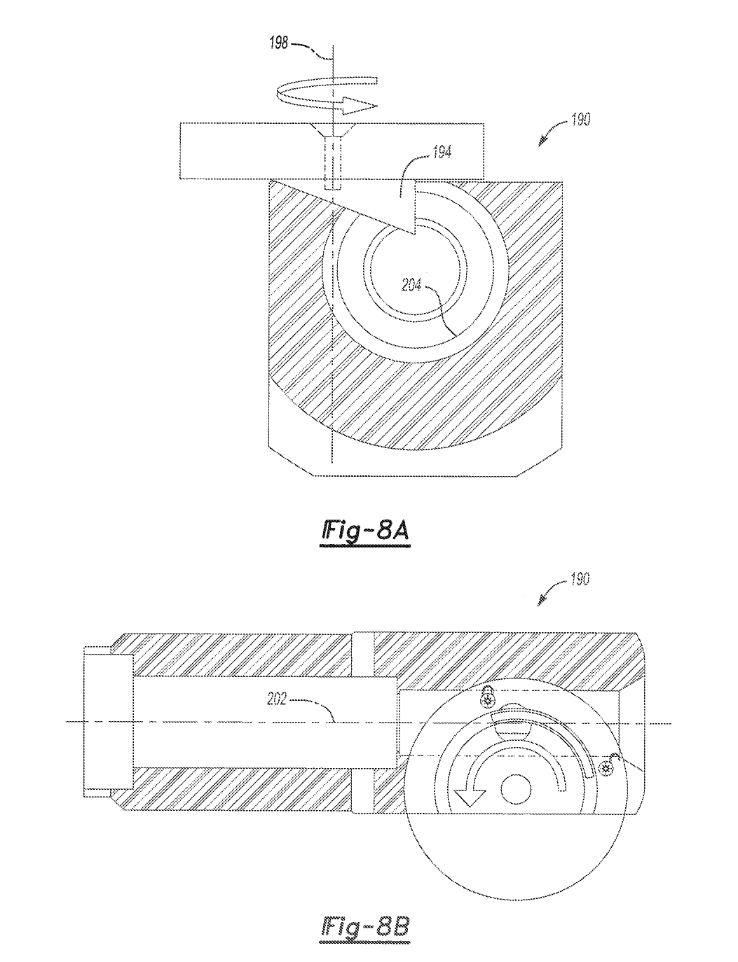

In another embodiment, the connector of the present invention can include a clamping member that includes a protrusion configured to press into a waveguide for clamping or securing the waveguide to the connector. Such protrusion can be moved (e.g., rotated) such that a clamping surface of the protrusion contacts the waveguide by virtue of movement of clamping member. The protrusion and/or clamping surface will then press against and/or potentially cut into the waveguide (e.g., a second waveguide) or jacket of the waveguide for securing and aligning the waveguide within the connector. Such movement of the clamping member can also press the waveguide against another clamping surface, for example, of the connector. With reference to FIGS. 8A and 8B, a connector 190 is illustrated to include a pivoting clamping member 192 having a protrusion 194 shown as a blade (e.g., a curved blade). As shown the clamping member 192 is pivotable about an axis 198 that is non-parallel and/or substantially perpendicular to an axis 202 of the connector 190, a waveguide that has been inserted in the connector or both. When pivoted, the protrusion 194 moves through a slot in the body of the connector 190. When the clamping member 192 is turned, the protrusion engages the waveguide (e.g., a second or disposable waveguide), such that a surface of the protrusion 194 presses against and potentially cuts into the waveguide (e.g., the jacket of the waveguide) and may also press the waveguide against a surface 204 of the connector 190 thereby holding the waveguide aligned and securely held. To remove or disengage the waveguide, the clamping member can be configured for rotation in a direction opposite the direction that originally clamped the waveguide. However, if the member 192 is provided with a ratchet feature so it can only rotate in one direction, then one particular single use provision can be provided. The protrusion 194 can have a benign profile at the bottom of the section where it starts to engage the waveguide and the clamping member 192 can be rotated until the waveguide is securely held. The clamping member can be configured to rotate further in the same direction driving a sharper section of the protrusion or clamping member into the waveguide and either crushing or cutting the waveguide (e.g., include the jacket and core), preferably without severing the end off. In this fashion, the ability of waveguide to be clamped in place or propagate light can be hindered thereby encouraging single use of the waveguide.

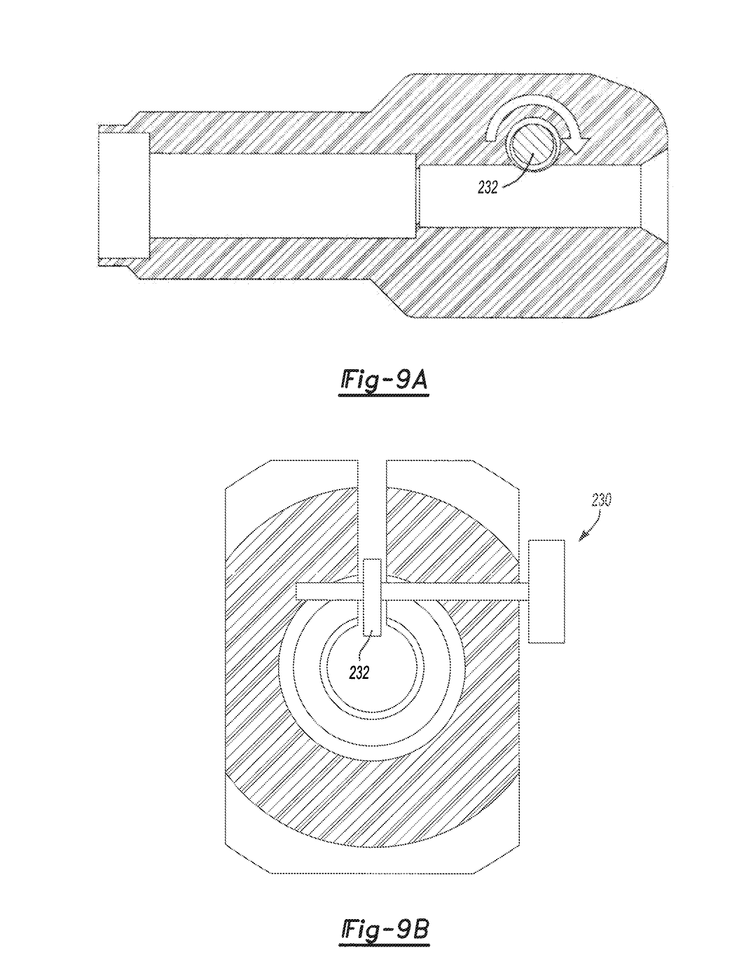

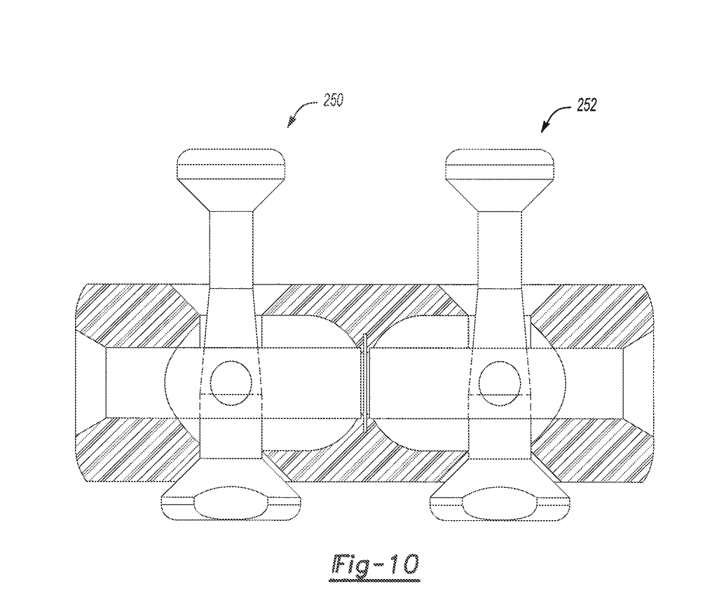

As another addition or alternative, with reference to FIG. 9, a connector can include a clamping member 230 fitted with an eccentric cam 232. Then, upon movement (e.g., rotation) of the clamping bar 230 about an axis thereof, a clamping surface can press against the waveguide such that the waveguide is pressed into a clamping surface of the connector. In this manner, the waveguide can be secured and aligned.

As another addition or alternative, with reference to FIG. 10, a connector can include a first clamping member 250 and a second clamping member 252 such as any of those clamping members described herein. In such an embodiment, it is possible to connect waveguides of any type described, however, it is particularly suitable for connecting two waveguides of the plastic or disposable type.

It is to be understood that the connectors of the present invention can be formed of a variety of materials and can be formed used a variety of techniques. Suitable materials include polymeric material (e.g., glass filled epoxy, plastic, etc . . . ), metal, glass, combinations thereof or the like. Depending upon the material used, the connector can be formed by, molding, machining, a combination thereof or the like.

In each of the embodiments, there may be a visual indication of the clamping status of at least the second waveguide. For the embodiment of FIGS. 1-2, the connector 10 includes an opening 270 (e.g., through-hole) such that the clamping member 60 can be seen. When the waveguide is in the clamped or attached position, a visual indicator 272 (e.g., a line, color coding or the like) can be seen through the opening. Similar or same types of visual indicators may be used with the rest of the embodiments as well. As an alternative and as shown in FIGS. 6A, 6B and 8B, one or more visual indicators 280 can be covered and/or uncovered by the member or clamping bar during use of the connector for indicating attached or detached positions.

It will be recognized that the various embodiment of the present invention can each provide one or more of the following advantages: 1) disposability of waveguides that might otherwise need to be sterilized, which can lower cost; 2) the connector provides secure attachment to the waveguide while also allowing the waveguide to be later released, potentially through single hand operation; 3) the connector can have the ability to allow for larger manufacture tolerances due to the manner of engagement or clamping of the waveguide; 4) the connector can be formed of fewer parts allowing easier construction, manufacture or both; and/or 5) the connector can be engaged and disengaged with a single hand.

Unless stated otherwise, dimensions and geometries of the various structures depicted herein are not intended to be restrictive of the invention, and other dimensions or geometries are possible. Plural structural components can be provided by a single integrated structure. Alternatively, a single integrated structure might be divided into separate plural components. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention.

What is claimed is:

1. An assembly useful for performing photodynamic therapy, the assembly comprising:
a first waveguide or light component;
a connector having a first feature connecting the connector to the first waveguide or light component and a second feature for connecting the connector to a second waveguide, wherein:
i. the second feature includes an opening for receiving an end of the second waveguide;
ii. the second feature includes a clamping member that clamps the second waveguide between a first surface and a second surface of the connector for aligning the first waveguide or light component with the second waveguide such that light can be communicated therebetween.

2. An assembly as in claim 1 wherein the second waveguide is located within the opening of the second feature and an end of the second waveguide is clamped between the first surface and the second surface.

3. An assembly as in claim 1 wherein the first waveguide or light component is connected to a light source.

4. An assembly as in claim 2 wherein the second waveguide includes a second optical fiber disposed within a covering wherein:
i. the second optical fiber is formed of a polymeric material; and
ii. the covering is formed of a polymeric material.

5. An assembly as in claim 1 wherein the clamping member has an axis and the axis of the clamping member extends laterally with respect to an axis of the connector.

6. An assembly as in claim 5 wherein movement of the clamping member along its axis causes the first surface of the connector to move closer or further away from the second surface of the connector such that the second waveguide is securely, but releasably, clamped between the first surface and the second surface.

7. An assembly as in claim 1 wherein the first feature includes an opening into which an end of the first waveguide extends.

8. An assembly as in claim 7 wherein the end of the first waveguide is securely located within the opening of the first feature by virtue of an interference fit between the connector and the first waveguide.

9. An assembly as in claim 6 wherein the first surface is disposed at an angle of at least 1° but less than 45° relative to the axis of the clamping member.

10. An assembly as in claim 9 wherein the clamping member has a cross-sectional area that progressively increases for forming the first clamping surface of the clamping member.

11. An assembly useful for performing photodynamic therapy, the assembly comprising:
a first waveguide, the first waveguide including a first optical fiber;
a second waveguide, the second waveguide including a second optical fiber disposed within a covering wherein:
i. the second optical fiber is formed of a polymeric material; and
ii. the covering is formed of a polymeric material, the polymeric material of the covering being polyethylene;
a connector having a first feature for connecting to the first waveguide and a second feature for connecting to the second waveguide, wherein:
i. the first feature includes an opening into which an end of the first waveguide extends;
ii. the end of the first waveguide is securely located within the opening of the first feature by virtue of an interference fit between the connector and the first waveguide;
iii. the second feature includes an opening into which an end of the second waveguide extends; and
iv. the second feature includes a clamping member that clamps the second waveguide between a first surface and a second surface of the connector for aligning the first optical fiber with the second optical fiber such that light can be communicated therebetween;
v. the clamping member has an axis and the axis of the clamping member extends laterally with respect to an axis of the connector;
vi. movement of the clamping member along its axis causes the first surface of the connector to move closer and further away from the second surface of the connector such that the second optical fiber is securely, but releasably, clamped between the first surface and the second surface.

12. An assembly as in claim 11 wherein the first waveguide is connected to a light source and the light source provides light to the first waveguide at wavelength suitable for performing photodynamic therapy.

13. A method of performing photodynamic therapy, the method comprising:
providing a connector connected to a first waveguide or light component using a first feature;
providing a second waveguide, the second waveguide being formed of a polymeric material;
connecting an end of the second waveguide to the connector such that the second waveguide is aligned with the first waveguide for receiving light from the first waveguide, the end of the second waveguide being clamped between a first surface and a second surface of the connector; and
directing light from the second waveguide at tissue for killing microbes.

14. A method as in claim 13 wherein the first surface of the connector is provided by a clamping member of the connector, the clamping member being rotatable relative to or moveable along an axis of the clamping member for moving the first surface and second surface toward each other.

15. A method as in claim 13 further comprising:
removing the second waveguide from the connector and attaching a third waveguide in its place; and
directing light at tissue for killing microbes.
